(19) United States
(12) Patent Application Publication
(10) Pub. No.: US 2018/0271434 A1
(43) Pub. Date: Sep. 27, 2018

(54) MATTRESS

(71) Applicant: A.H. Beard Pty. Ltd., Padstow (AU)

(72) Inventors: Brent Tyrrell, Summer Hill (AU); Justin Westrop, Hinchinbrook (AU); Jonathan Mata'afa, Ruse (AU); Tracy Duhring, Minto (AU); Sasha Lajmanovski, Mortdale (AU); Neville Middleton, Engadine (AU)

(73) Assignee: A.H Beard Pty. Ltd., Padstow (AU)

(21) Appl. No.: 15/915,331

(22) Filed: Mar. 8, 2018

(30) Foreign Application Priority Data
Mar. 13, 2017 (AU) 2017900863

(51) Int. Cl. A61B 5/00; A47C 21/00

(52) U.S. Cl. CPC A61B 5/4809; A47C 27/15; A61B 5/6892; A47C 21/00

(57) ABSTRACT

A mattress construction includes a top panel defining a sleep surface, a core layer disposed below the top panel and a peripheral border extending around the periphery of the top panel. The peripheral border having a first longitudinal segment, a second longitudinal segment, a first lateral segment and a second lateral segment. An outer surface is partially defined by the top panel and peripheral border. Included is a sleep tracker receiving arrangement. Each arrangement has a first aperture located in the outer surface in or adjacent the first longitudinal segment and a first sleeve disposed between the top panel and the core layer. The first sleeve having a first opening aligned with the first aperture. The arrangement is configured to removably receive a first sleep tracker through the first aperture and the first opening such that the first sleep tracker is at least substantially disposed within the first sleeve.

### REFERENCE TO RELATED APPLICATIONS

This application claims priority to Australian Provisional Application No. 2017900863, filed Mar. 13, 2017, which is hereby incorporated by reference in its entirety.

### FIELD OF THE INVENTION

The present invention relates to a mattress configure for use with a sleep tracker. The present invention also relates to a mattress system having a mattress and at least one sleep tracker.

### BACKGROUND OF THE INVENTION

Sleep trackers for monitoring sleep biometrics of a person are known. They can be used to track heart rate, breathing, snoring, sleep time, restless, time out of bed, time spent in light or deep sleep and many other sleep biometrics. When in use, current sleep trackers are typically positioned on top of the mattress or underneath the mattress itself.

A disadvantage of placing the sleep tracker on top of the mattress is that a person can feel the sleep tracker when sleeping, which may cause some discomfort and therefore reduce the quality of sleep. Further, it is difficult to secure the sleep tracker in place on top of the mattress, meaning that the sleep tracker is susceptible to being moved by a person. If the sleep tracker is moved during use, the quality of the measurements obtained by the sleep tracker may be reduced.

A disadvantage of placing the sleep tracker below the mattress is that the accuracy of the sleep tracker is significantly reduced, thereby reducing the quality of the measurements obtained by the sleep tracker.

### OBJECT OF THE INVENTION

It is an object of the present invention to overcome or at least ameliorate one or more of the foregoing disadvantages.

### SUMMARY OF THE INVENTION

In a first aspect, the present invention provides a mattress comprising:

a top panel defining a sleep surface;

a core layer disposed below the top panel;

a peripheral border extending from and around the periphery of the top panel, the peripheral border having a first longitudinal segment, a second longitudinal segment, a first lateral segment and a second lateral segment;

an outer surface at least partially defined by the top panel and peripheral border;

at least one sleep tracker receiving arrangement, each arrangement having:
a first aperture located in the outer surface, the first aperture located in or adjacent the first longitudinal segment; and
a first sleeve disposed between the top panel and the core layer, the first sleeve having a first opening aligned with the first aperture, and
   the arrangement configured to removably receive a first sleep tracker through the first aperture and the first opening such that the first sleep tracker is at least substantially disposed within the first sleeve.

In a preferred form, the at least one sleep tracker arrangement comprises a first arrangement and a second arrangement, and the first arrangement is disposed towards the first lateral segment and the second arrangement is disposed towards the second lateral segment.

In a preferred form, for at least one of the arrangements, the first sleeve extends from the first longitudinal segment towards the second longitudinal segment.

In a preferred form, for at least one of the arrangements, the first sleeve extends from the first longitudinal segment to the second longitudinal segment.

In a preferred form, for at least one of the arrangements, the arrangement further comprises a second aperture located in the outer surface, the second aperture located in or adjacent the second longitudinal segment, and the first sleeve further comprises a second opening aligned with the second aperture, wherein the first sleep tracker may be removably received through the first aperture and first opening of the first sleeve or through the second aperture and second opening of the first sleeve such that the first sleep tracker is at least substantially received within the first sleeve.

In a preferred form, for at least one of the arrangements, the first aperture is in the first longitudinal segment and the second aperture is in the second longitudinal segment, and the first aperture is at least substantially aligned with the second aperture.

In a preferred form, for at least one of the arrangements, the arrangement is configured to receive two sleep trackers therein, the first sleep tracker through the first aperture and the first opening of the first sleeve, and a second sleep tracker through the second aperture and second opening of the first sleeve.

In a preferred form, for at least one of the arrangements, the arrangement further comprises:

a second aperture located in the outer surface, the second aperture located in or adjacent the second longitudinal segment; and

a second sleeve disposed between the top panel and the core layer, the second sleeve having a first opening aligned with the second aperture,

wherein the arrangement is configured to removably receive a second sleep tracker through the second aperture and the second opening such that the second sleep tracker is at least substantially disposed within the second sleeve.

In a preferred form, for at least one of the arrangements, the second sleeve extends from the second longitudinal segment towards the first longitudinal segment.

In a preferred form, the mattress further comprises a comfort layer disposed between the top panel and the core layer.

In a preferred form, each sleeve is disposed between the top panel and the comfort layer.

In a preferred form, the mattress further comprises an internal frame extending around the periphery of the core layer between the core layer and the peripheral border, wherein each sleeve extends between the frame and the top panel.

In a preferred form, wherein each sleeve is bonded to the frame.

In a preferred form, each sleeve further comprises at least one flap that is bonded to the frame.

According to an embodiment, there is provided a mattress system comprising:

a mattress according to the first aspect and/or any of the preferred forms above; and

at least one sleep tracker removably received in at least one of the sleep tracker receiving arrangements.

According to an embodiment, there is provided a method of tracking sleep of a user, the method comprising the steps of:

inserting a sleep tracker into a sleep tracking receiving arrangement of the first aspect and/or any of the preferred forms above;

operating the sleep tracker to track the sleep of a user sleeping on the sleep surface of the mattress.

### BRIEF DESCRIPTION OF THE DRAWINGS

A preferred embodiment of the invention will be described hereinafter, by way of example only, with reference to the accompanying drawings, wherein:

FIG. 1 is a perspective view of a mattress according to a first embodiment.

FIG. 2 is an exploded parts perspective view of the mattress of FIG. 1.

FIG. 3 is an enlarged partial view of a sleeve highlighted by Region A of FIG. 2.

FIG. 4 is a cross section of the sleeve of FIG. 3 taken along the line B-B.

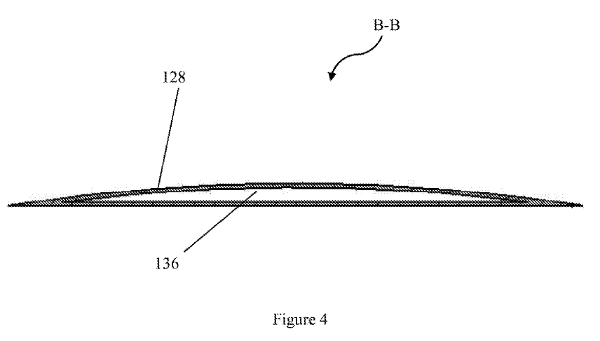

FIG. 5 is an enlarged partial view of an aperture and eyelet highlighted by Region C of FIG. 2 omitting a sleep tracker.

FIG. 6 is an enlarged partial view of an aperture and eyelet highlighted by Region C of FIG. 2 including a sleep tracker.

FIG. 7 is a schematic representation of the mattress of FIG. 1 having two sleep trackers received therein.

FIG. 8 is a schematic representation of a mattress according to a second embodiment;

FIG. 9 is a schematic representation of a mattress according to a third embodiment.

FIG. 10 is a schematic representation of a mattress according to fourth embodiment.

FIG. 11 is a schematic representation of a mattress according to fifth embodiment.

### DESCRIPTION OF THE EMBODIMENTS

For the purposes of promoting understanding of the principles of the invention, reference will now be made to the embodiments illustrated in the drawings and specific language will be used to describe the same. It will nevertheless be understood that no limitation of the scope of the invention is hereby intended and alterations and modifications in the devices, systems and representations illustrated in the Figures of the drawing, and further applications of the principles of the present invention as illustrated herein being contemplated as would normally occur to one skilled in the art to which the invention relates.

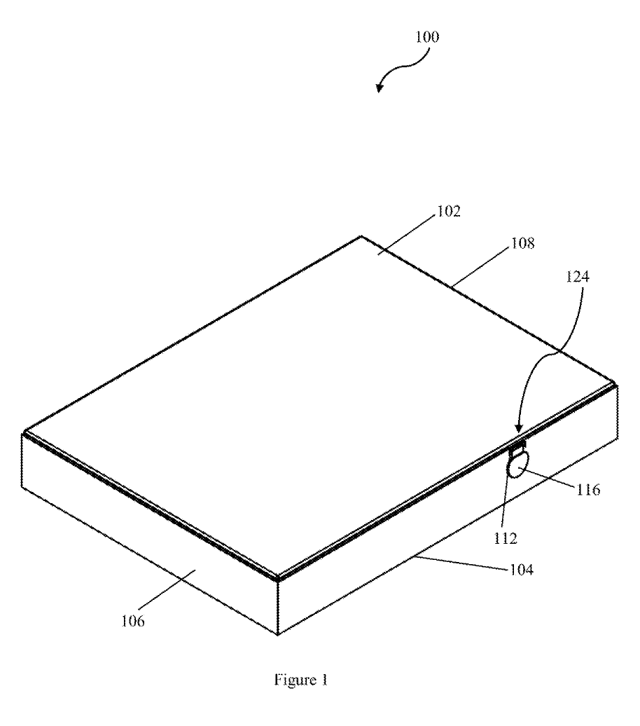

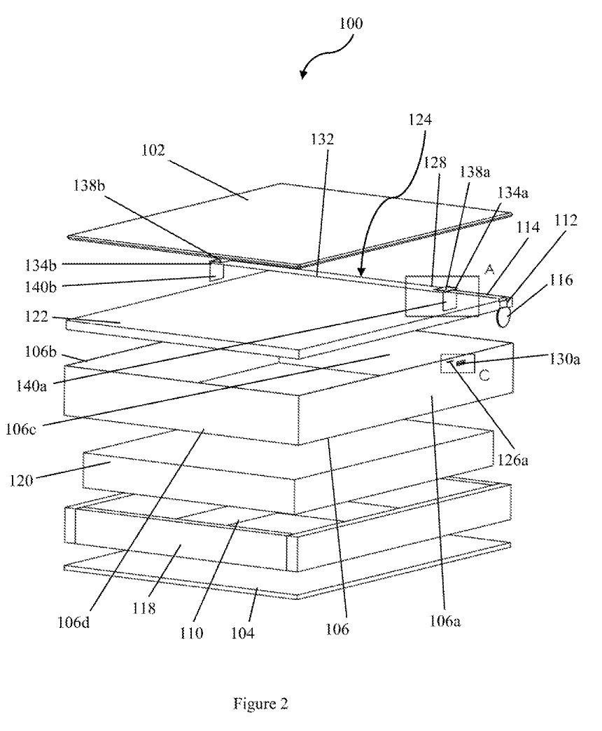

FIG. 1 shows a mattress 100 according to a first embodiment. The mattress 100 has a top panel 102 defining a sleep surface, a base panel 104 and a peripheral border 106. Referring to FIG. 2, the peripheral border 106 comprises a pair of longitudinal segments 106a and 106b, a lateral segment 106c forming the head end of the mattress 100, and a lateral segment 106d forming the foot end of the mattress 100. The lateral segment 106c and the lateral segment 106d extend between the longitudinal segments 106a and 106b. The top panel 102 is disposed above the base panel 104, and the peripheral border 106 extends between and around the periphery of the top panel 102 and the base panel 104. The top panel 102, base panel 104 and peripheral border 106 together define an outer surface 108 (see FIG. 1) and an interior 110 of the mattress 100.

The mattress 100 is configured for use with one or more sleep trackers 112, thereby forming a mattress system. The sleep tracker 112 comprises an elongate sensing segment 114 and a computing portion 116. An example sleep tracker 112 suitable for use with the mattress 100 is the RestOn tracker from Sleepace, however it is envisaged that other sleep trackers may be used with the mattress 100.

The mattress 100 further comprises a frame 118, a core layer 120, a comfort layer 122 and a sleep tracking receiving arrangement 124. The frame 118 is disposed within the interior 110 above the base panel 104 and is used to retain the structural shape of the mattress 100. The frame 118 is configured to receive the core layer 120 therein such that the frame 118 surrounds the periphery of the core layer 120, and the comfort layer 122 is disposed between the core layer 120 and the top panel 102.

The frame 118 is made from a rigid foam material, however, it is envisaged that the frame 118 may be made from other suitable materials known in the art. The core layer 120 may be, for example, a foam block, a pocket spring assembly as used in traditional mattresses, or an inflatable fluid bladder assembly as used in air bed mattresses such as the Sleepsense® by A.H. Beard®. It is also envisaged that the core layer 120 may be any suitable material/assembly known in the art. The comfort layer 122 may comprise, for example, an outer tick layer, a fibre fill layer, a foam layer, and a fibrous quilt backing layer. These layers of the comfort layer 122 are adhered or stitched together. The tick layer is typically tufted and may comprise, for example, polyester, cotton and/or polypropylene fabric. The fibre fill layer will typically comprise polyester and/or wool. It is also envisaged that the comfort layer 122 may comprise other suitable materials and combinations known in the art.

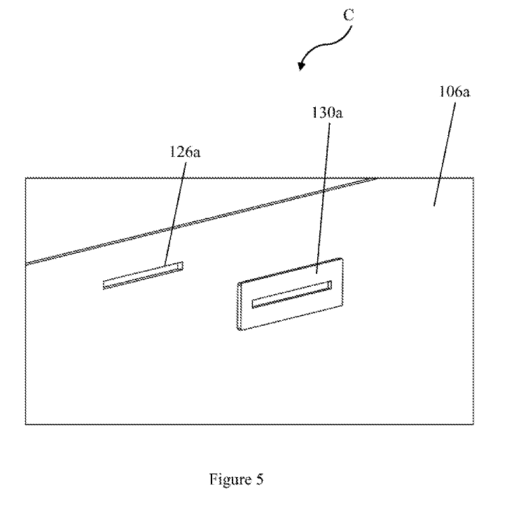

The sleep tracker receiving arrangement 124 comprises a first aperture 126a located in the longitudinal segment 106a and a sleeve 128. Although not illustrated, the sleep tracker receiving arrangement 124 further comprises a second aperture 126b in the longitudinal segment 106b aligned with and opposite to the first aperture 126a. Alternatively, the first aperture 126a and the second aperture 126b may be located in the outer surface 108 adjacent the longitudinal segment 106a and the longitudinal 106b respectively, or through stitching that joins the top panel 102 to the peripheral border 106. Referring to FIG. 5, associated with the first aperture 126a is an eyelet 130a, and associated with the second aperture 126b is an eyelet 130b. The eyelets 130a and 130b support and protect the first aperture 126a and the second aperture 126b respectively.

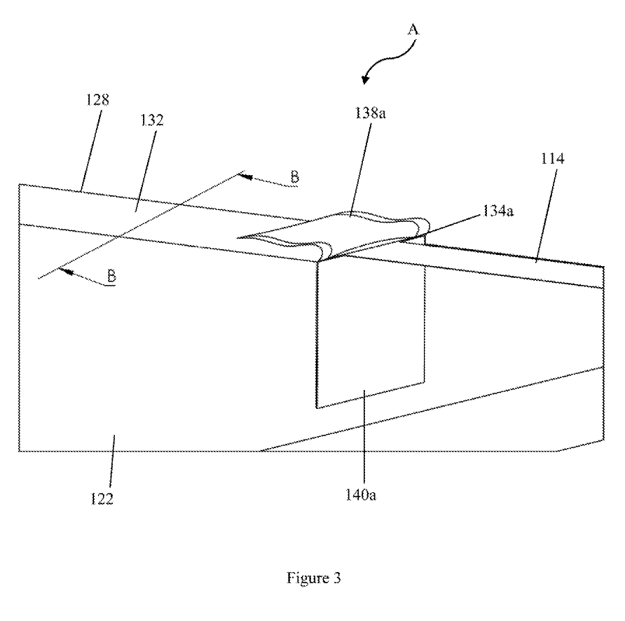

The sleeve 128 has an elongate portion 132, a first opening 134a, a second opening 134b and an interior 136 (see FIG. 5). The interior 136 is configured to receive the sensing segment 114 of the sleep tracker 112 therein. Referring to FIG. 3, the sleeve 128 has a first top flap 138a and a first bottom flap 140a defining the first opening 134a. The first top flap 138a is folded back onto and secured to the elongate portion 132 of the sleeve 128, and the first bottom flap 140a is bonded to the frame 118 thereby securing the sleeve 128 in position within the mattress 100. Similarly, referring to FIG. 2, the sleeve 128 has a second top flap 138b and a second bottom flap 140b defining the first opening 134b. The second top flap 138b is folded back onto and secured to the elongate portion 132 of the sleeve 128, and the second bottom flap 140b is bonded to the frame 118 thereby securing the sleeve 128 in position within the mattress 100. Securing the sleeve 128 in position within the mattress 100 ensures that any sleep tracker 112 received therein does not move when in use, which would otherwise reduce the quality of the measurements obtained from the sleep tracker 112.

The sleeve 128 is disposed towards the lateral segment 106c (i.e. the head of the mattress 100) between the top panel 102 and the core layer 120, extends between the longitudinal segments 106a and 106b of the peripheral border 106, and extends over the frame 118 and core layer 120. However, in a preferred embodiment, and as illustrated in the accompanying drawings, the sleeve 128 is disposed between the top panel 102 and the comfort layer 122. It is also envisaged that the sleeve 128 may be disposed between the core layer 120 and the comfort layer 122. When the mattress 100 is assembled, the first opening 134a of the sleeve 128 is aligned with the first aperture 126a, and the second opening 134b of the sleeve 128 is aligned with the second aperture 126b.

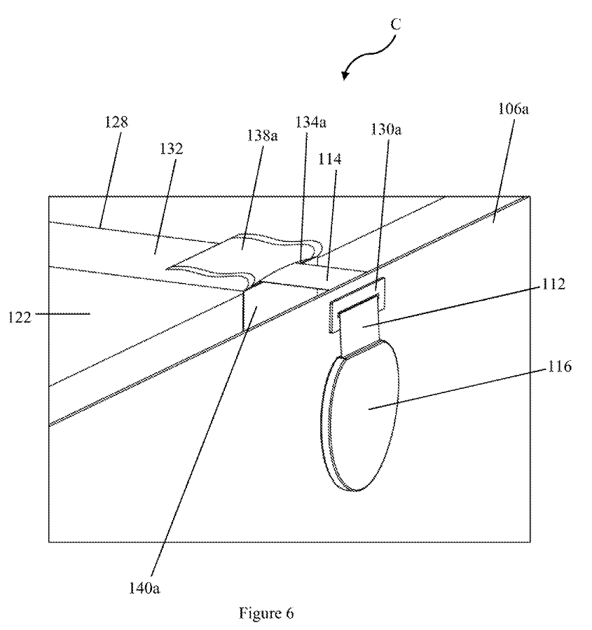

Inserting a sleep tracker 112 into the mattress 100 will now be discussed. Referring to FIG. 6, inserting the sleep tracker 112 into the mattress 100 involves passing the elongate sensing segment 114 of the sleep tracker 112 through the first aperture 126a such that the elongate sensing segment 114 extends through the first opening 134a and is received within the interior 136 of the sleeve 128. Referring to FIG. 1, when the sleep tracker 112 is inserted into the mattress 100 through first aperture 126a, the computing portion 116 remains external to the mattress 100 and hangs down substantially flush with the longitudinal segment 106a of the peripheral border 106. Removing the sleep tracker 112 from the mattress 100 involves simply pulling the sleep tracker 112 out from the interior 136 of the sleeve 128 through the first opening 134a and first aperture 126a.

Alternatively, the sleep tracker 112 may be inserted into the mattress 100 by passing the elongate sensing segment 114 of the sleep tracker 112 through the second aperture 126b such that the elongate sensing segment 114 extends through the second opening 134b and is received within the interior 136 of the sleeve 128. When the sleep tracker 112 is inserted into the mattress 100 through second aperture 126b, the computing portion 116 remains external to the mattress 100 and hangs down substantially flush with the longitudinal segment 106b of the peripheral border 106. Removing the sleep tracker 112 from the mattress 100 involves simply pulling the sleep tracker 112 out from the interior of the sleeve 128 through the second opening 134b and second aperture 126b.

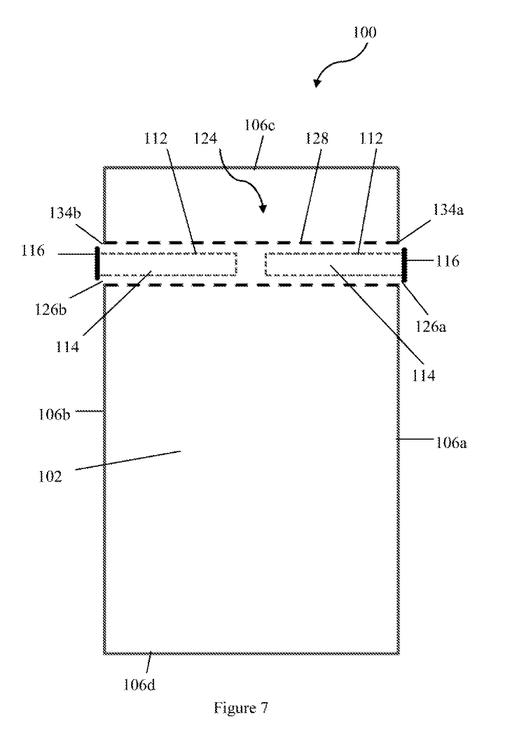

Although the mattress 100 has been described above with a single sleeve 128 receiving a single sleep tracker 112 therein, referring to FIG. 7, it is also envisaged that the sleeve 128 may be configured to receive two sleep trackers 108 in the interior 136, one sleep tracker 112 through the first aperture 126a and the first opening 134a and the another sleep tracker 112 through the second aperture 126b and the second opening 134b. In this case, the elongate sensing segments 114 of each of the sleep trackers 112 would only extend a distance into the interior 136 of the sleeve 128 typically so as not to overlap with each other.

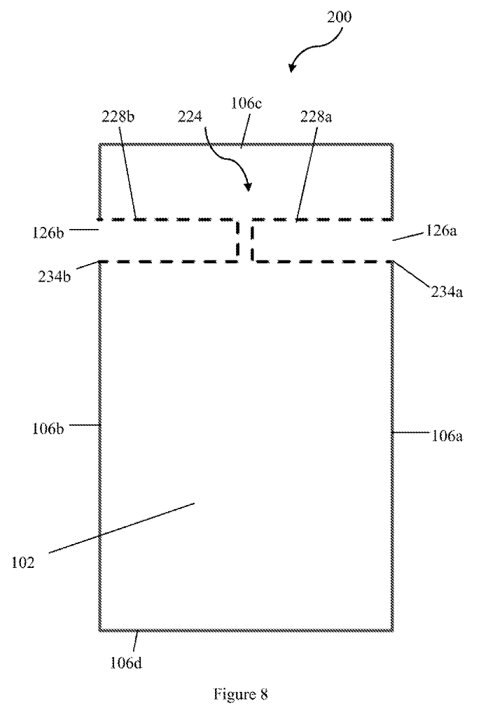

FIG. 8 is a schematic representation of a mattress 200 according to a second embodiment. The mattress 200 is similar to the mattress 100, except that the sleep tracker receiving arrangement 224 of the mattress 200 comprises a first sleeve 228a and a second sleeve 228b. The first sleeve 228a comprises an opening 234a aligned with the first aperture 126a, and the second sleeve 228b comprises an opening 234b aligned with the second aperture 126b. Each of the sleeves 228a and 228b are configured to receive a sleep tracker 112 therein (i.e. one sleep tracker 112 for each side of the mattress 200).

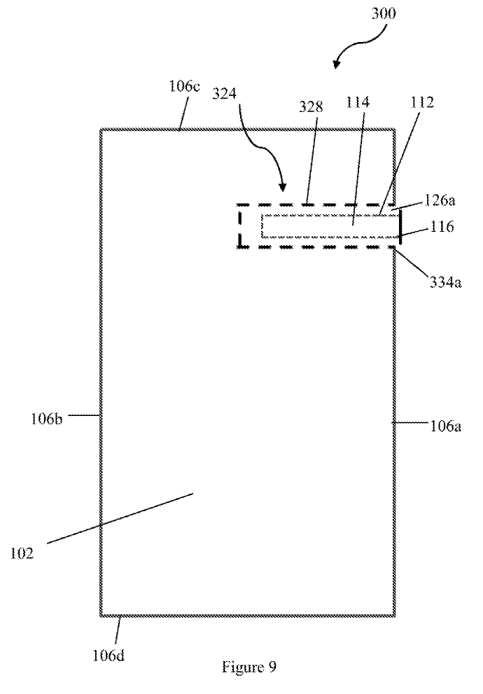

FIG. 9 is a schematic representation of a mattress 300 according to a third embodiment. The mattress 300 is similar to the mattress 200, except that the sleep tracker receiving arrangement 324 only has a single sleeve 328 that extends from the longitudinal segment 106a towards the longitudinal segment 106b. The sleeve 328 may extend across the entire mattress 200 (i.e. across both sides of the mattress 300), or only partially across the mattress (i.e. only one side of the mattress 300). The sleeve 328 has an opening 334 aligned with the first aperture 126a such that a sleep tracker 112 may be received in the sleeve 328.

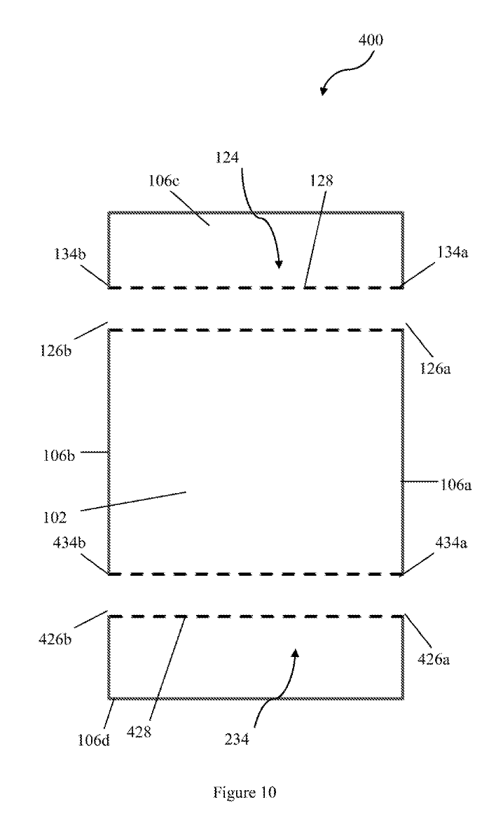

FIG. 10 is a schematic representation of a mattress 400 according to a fourth embodiment. The mattress 400 is similar to the mattress 100, except that the mattress 400 includes a second sleep tracker receiving arrangement 424 disposed towards the lateral segment 106d (i.e. the foot end of the mattress 400). The second sleep tracker receiving arrangement 424 is identical to the sleep tracker receiving arrangement 124 of mattress 100. The second sleep tracker receiving arrangement 424 comprises a first aperture 426a located in the longitudinal segment 106a of the peripheral border 106 and a second aperture 426b located in the longitudinal segment 106b of the peripheral border 106. The second sleep tracker receiving arrangement 424 further comprises a sleeve 428 having a first opening 434a aligned with the aperture 426a and a second opening 434b aligned with the aperture 426b. The second sleep tracker receiving arrangement 424 is configured to receive a single sleep tracker 112 therein as described above with respect to the sleep tracker receiving arrangement 124. It is also envisaged that the sleeve 424 may be configured to receive two sleep trackers 112 as described in paragraph [0045].

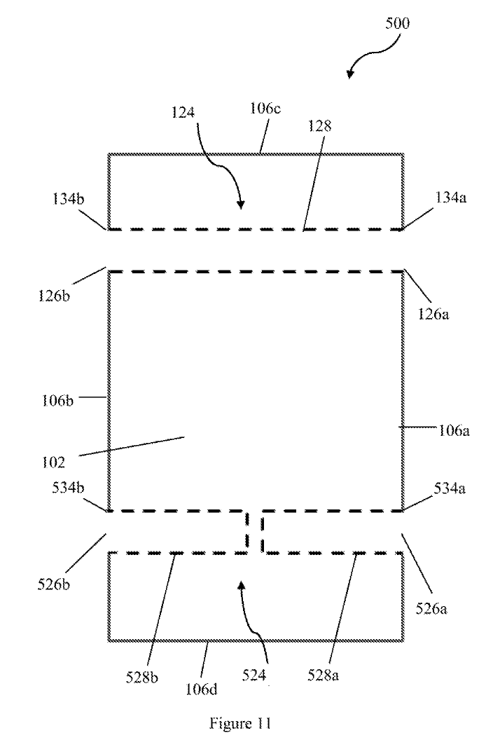

FIG. 11 is a schematic representation of a mattress 500 according to a fifth embodiment. The mattress 500 is similar to the mattress 100, except that the mattress 500 further includes a second sleep tracker sleeping arrangement 524 disposed towards the lateral segment 106d (i.e. foot end of the mattress 500). The second sleep tracker receiving arrangement 524 is identical to the sleep tracker sleeping arrangement 224 of mattress 200. The second sleep tracker receiving arrangement 524 comprises a first aperture 526a located in the longitudinal segment 106a of the peripheral border 106 and a second aperture 526b located in the longitudinal segment 106b of the peripheral border 106. The first sleeve 528a has an opening 534a aligned with the aperture 526a, and the second sleeve 528 has an opening 534b aligned with the aperture 526b. Each of the sleeves 528a and 528b are configured to receive a sleep tracker 112 therein (i.e. one sleep tracker 112 for each side of the mattress 500).

It is also envisaged that a mattress may comprises any combination of the sleep tracker receiving arrangements described above. It is also envisaged that a mattress may comprises 3 or more of the sleep tracker receiving arrangements described above as desired.

The sleep of a user may be tracked by using any of the above described mattresses by inserting a sleep tracker 112 into a sleep tracker receiving arrangement of a respective mattress and operating the sleep tracker 112 so as to track the sleep of a user sleeping on the sleep surface of the respective mattress.

As the sleep tracker 112 is disposed towards the top panel, the accuracy of the measurements obtained from the sleep tracker 112 is not reduced significantly. Further, as the sleep tracker 112 is not disposed directly on the top panel 102, any discomfort caused by the sleep tracker 112 is reduced. Another advantage of the mattresses described above, is that movement of the sleep tracker 112 during use is reduced thereby increasing the quality and reliability of the measurements obtained from the sleep tracker 112.

Further, being able to removably insert a sleep tracker 112 into any of the mattresses described above allows the sleep tracker 112 to be replaced if faulty or upgraded when newer sleep trackers become available. It is also envisaged that any of the mattress described above may be purchased without a sleep tracker such that a user may purchase a sleep tracker of their choosing, or so that the user may retrofit a sleep tracker at a later stage.

Although the mattress has been described with reference to a preferred embodiment, it will be appreciated by persons skilled in the art that the invention may be embodied in many other forms.

While the invention has been illustrated and described in detail in the drawing and foregoing description, the same is to be considered as illustrative and not restrictive in character, it being understood that only the preferred embodiment has been shown and described and that all changes, modifications and equivalents that come within the spirit of the inventions disclosed are desired to be protected. The articles “a”, “an”, “said” and “the” are not limited to a singular element, and include one or more such elements.

1. A mattress comprising:
a top panel defining a sleep surface;
a core layer disposed below the top panel;
a peripheral border extending from and around the periphery of the top panel, the peripheral border having a first longitudinal segment, a second longitudinal segment, a first lateral segment and a second lateral segment;
an outer surface at least partially defined by the top panel and peripheral border; and
at least one sleep tracker receiving arrangement, each arrangement having:
a first aperture located in the outer surface, the first aperture located in or adjacent the first longitudinal segment; and
a first sleeve disposed between the top panel and the core layer, the first sleeve having a first opening aligned with the first aperture, and
the arrangement configured to removably receive a first sleep tracker through the first aperture and the first opening such that the first sleep tracker is at least substantially disposed within the first sleeve.

2. The mattress of claim 1, wherein the at least one sleep tracker arrangement comprises a first arrangement and a second arrangement, and the first arrangement is disposed towards the first lateral segment and the second arrangement is disposed towards the second lateral segment.

3. The mattress of claim 1, wherein, for at least one of the arrangements, the first sleeve extends from the first longitudinal segment towards the second longitudinal segment.

4. The mattress of claim 1, wherein, for at least one of the arrangements, the first sleeve extends from the first longitudinal segment to the second longitudinal segment.

5. The mattress of claim 4, wherein, for at least one of the arrangements, the arrangement further comprises a second aperture located in the outer surface, the second aperture located in or adjacent the second longitudinal segment, and the first sleeve further comprises a second opening aligned with the second aperture, wherein the first sleep tracker may be removably received through the first aperture and first opening of the first sleeve or through the second aperture and second opening of the first sleeve such that the first sleep tracker is at least substantially received within the first sleeve.

6. The mattress of claim 5, wherein, for at least one of the arrangements, the first aperture is in the first longitudinal segment and the second aperture is in the second longitudinal segment, and the first aperture is at least substantially aligned with the second aperture.

7. The mattress of claim 5, wherein, for at least one of the arrangements, the arrangement is configured to receive two sleep trackers therein, the first sleep tracker through the first aperture and the first opening of the first sleeve, and a second sleep tracker through the second aperture and second opening of the first sleeve.

8. The mattress of claim 1, wherein, for at least one of the arrangements, the arrangement further comprises:
a second aperture located in the outer surface, the second aperture located in or adjacent the second longitudinal segment; and
a second sleeve disposed between the top panel and the core layer, the second sleeve having a first opening aligned with the second aperture,
wherein the arrangement is configured to removably receive a second sleep tracker through the second aperture and the second opening such that the second sleep tracker is at least substantially disposed within the second sleeve.

9. The mattress of claim 8, wherein, for at least one of the arrangements, the second sleeve extends from the second longitudinal segment towards the first longitudinal segment.

10. The mattress of claim 1, further comprising a comfort layer disposed between the top panel and the core layer.

11. The mattress of claim 10, wherein each sleeve is disposed between the top panel and the comfort layer.

12. The mattress of claim 1, further comprising an internal frame extending around the periphery of the core layer between the core layer and the peripheral border, wherein each sleeve extends between the frame and the top panel.

13. The mattress of claim 1, wherein each sleeve is bonded to the frame.

14. The mattress of claim 13, wherein each sleeve comprises at least one flap that is bonded to the frame.

15. The mattress of claim 2, wherein, for at least one of the arrangements, the first sleeve extends from the first longitudinal segment to the second longitudinal segment.

16. The mattress of claim 15, wherein, for at least one of the arrangements, the arrangement further comprises a second aperture located in the outer surface, the second aperture located in or adjacent the second longitudinal segment, and the first sleeve further comprises a second opening aligned with the second aperture, wherein the first sleep tracker may be removably received through the first aperture and first opening of the first sleeve or through the second aperture and second opening of the first sleeve such that the first sleep tracker is at least substantially received within the first sleeve.

17. The mattress of claim 16, wherein, for at least one of the arrangements, the first aperture is in the first longitudinal segment and the second aperture is in the second longitudinal segment, and the first aperture is at least substantially aligned with the second aperture.

18. The mattress of claim 17, wherein, for at least one of the arrangements, the arrangement is configured to receive two sleep trackers therein, the first sleep tracker through the first aperture and the first opening of the first sleeve, and a second sleep tracker through the second aperture and second opening of the first sleeve.

19. A mattress system comprising:
a mattress according to claim 1; and
at least one sleep tracker removably received in at least one of the sleep tracker receiving arrangements.

20. A method of tracking sleep of a user, the method comprising the steps of:
inserting a sleep tracker into a sleep tracking receiving arrangement of according to claim 1; and
operating the sleep tracker to track the sleep of a user sleeping on the sleep surface of the mattress.
